(19) United States
(12) Patent Application Publication
(10) Pub. No.: US 2026/0199419 A1
(43) Pub. Date: Jul. 16, 2026

(54) NON-THERAPEUTIC USE OF AN ARONIA BERRY EXTRACT

(71) Applicant: BIOACTOR BV, Maastricht (NL)

(72) Inventors: Antonie Johannes Van Der Saag, Maastricht (NL); Yala Stevens, Maastricht (NL); Sanne Ahies, Maastricht (NL)

(21) Appl. No.: 19/134,471

(22) PCT Filed: Nov. 30, 2023
(86) PCT No.: PCT/EP2023/083708

§ 371 Date: May 30, 2025

(30) Foreign Application Priority Data
Dec. 1, 2022 (EP) 22210800.3

(51) Int. Cl. A61K 36/73; A61K 31/122; A61K 31/145; A61K 31/205; A61K 31/355; A61K 31/375; A61K 31/385; A61K 31/522; A61K 31/714; A61K 33/30; A61K 33/34; A61P 27/04

(52) U.S. Cl. CPC A61K 36/73; A61K 31/122; A61K 31/145; A61K 31/205; A61K 31/355; A61K 31/375; A61K 31/385; A61K 31/522; A61K 31/714; A61K 33/30; A61K 33/34; A61P 27/04

(57) ABSTRACT

The present invention relates to the field of food supplementation, in particular to the field of nutraceuticals, more in particular to the use of an Aronia berry extracts and compositions thereof. More specifically, the present invention relates to the non-therapeutic use of an Aronia berry extract or composition comprising such extract for improving ocular blood flow, improving symptoms of dry eyes, and/or improving symptoms of subjective eye fatigue in a human subject.

### FIELD OF THE INVENTION

The present invention relates to the field of food supplementation, in particular to the field of nutraceuticals, more in particular to the use of an Aronia berry extracts and compositions thereof. More specifically, the present invention relates to the non-therapeutic use of an Aronia berry extract or composition comprising such extract for improving eye parameters. In particular, the invention related to the non-therapeutic use of an Aronia berry extract or composition thereof for improving ocular blood flow, improving symptoms of dry eyes, and/or improving symptoms of subjective eye fatigue in a human subject.

### BACKGROUND TO THE INVENTION

Young adults and adults in general are a segment of the population that are prone to experiencing long periods of digital screen time due to online education, gaming and online working. Digital screen time among students and working professionals grows rapidly to 8-12 hours per day. Consequently, more and more individuals are dealing with fatigued eyes, leading to symptoms of dry eye or digital eye strain.

Accordingly, it was an object of the present invention to provide a solution to such problems. The inventors have now surprisingly found that Aronia berry extracts or compositions comprising the same are suitable for improving ocular blood flow, improving symptoms of dry eyes, and/or improving symptoms of subjective eye fatigue in a human subject, thereby providing a solution to some of the problems listed herein above.

It is known that Aronia berries contain a large amount of carotene and flavonoid (polyphenolic compounds) such as anthocyanins, luteins and zeaxanthins which have beneficial health effects. In particular, anthocyanins, which are found in a very high concentration in Aronia melanocarpa (black chokeberries), are associated with improved cognitive performance and vascular function in healthy middle-aged individuals and young adults (Ahles et al. 2020). Anthocyanins and their associated compounds, such as cyanidin-3-glycosides, are able to cross the blood-brain barrier. More recently, several studies have shown a potential for anthocyanin-rich dietary interventions to target various symptoms of eye strain and eye performance (Riva et al. 2017; Kan et al. 2020). Despite the beneficial health effects, intake of anthocyanins in the population is low. Supplementation of anthocyanin or natural components containing the flavonoid is an important approach to increase intake.

A detailed study of the effects of Aronia berry extracts on several aspects of eye fatigue, has not yet been performed until now, and it was thus unexpected that supplementation with Aronia berry extracts could improve symptoms of such eye fatigue.

### SUMMARY OF THE INVENTION

In a first aspect, the present invention provides the non-therapeutic use of an Aronia berry extract or a composition comprising said Aronia berry extract for improving ocular blood flow, improving symptoms of dry eyes, and/or improving symptoms of subjective eye fatigue in a human subject.

In a further embodiment, the improved ocular blood flow is the result of improved retinal vasculature, in particular improved retinal microvasculature.

In another embodiment, the dry eye symptoms are reduced tear production, increased tear osmolarity and/or tear film instability.

In yet another embodiment, the subjective eye fatigue symptoms are blurred vision at near distances, blurred vision at intermediate distances, blurred vision at far distances, difficulty or slowness in refocusing eyes from one distance to another, irritated eyes, burning eyes, dry eyes, eye strain, headache, tired eyes, and/or sensitivity to bright lights,

In a specific embodiment, the Aronia berry extract comprises at least 10% (w/w) anthocyanins, preferably at least 15% (w/w) anthocyanins, more preferably at least 20% (w/w) anthocyanins, even more preferably at least 25% (w/w) anthocyanins.

In another specific embodiment, the anthocyanin is a cyanidin-3-O-glycoside selected from the list comprising: cyanidin-3-galactoside, cyanidin-3-glucoside, cyanidin-3-arabinoside, cyanidin-3-xyloside or any combination thereof.

In yet another embodiment, in the case that cyanidin-3-galactoside is present in the Aronia berry extract or a composition comprising said Aronia berry extract, then cyanidin-3-galactoside comprises at least 30% (w/w), preferably at least 40% (w/w), more preferably at least 50% (w/w), most preferably at least 60% (w/w) of the total amount of cyanidin-3-O-glycoside.

In a particular embodiment, the Aronia berry extract comprises at least about 5% (w/w) cyanidin-3-galactoside, preferably at least about 10% (w/) cyanidin-3-galactoside, more preferably at least about 15% (w/w) cyanidin-3-galactoside (w/w) of the total amount of the extract.

In another particular embodiment, the composition of the present invention comprises about and between 1 mg to 1000 mg Aronia berry extract, such as about and between 15 mg to 750 mg, about and between 30 mg to 750 mg of Aronia berry extract, about and between 65-750 mg of Aronia berry extract, about and between 90-750 mg Aronia berry extract or about and between 65 and 500 mg Aronia berry extract.

In yet another particular embodiment, the composition comprises about and between 1 mg to 500 mg, preferably about and between 10 mg to 300 mg, more preferably about and between 15 mg to 200 mg of anthocyanin.

In an embodiment, the composition comprising the Aronia berry extract further comprises one or more compounds selected from a group consisting of: caffeine, taurine, theanine, inositol, guarana, magnesium, melatonin, ribose, carnitine, co-enzym Q10, alpha-glycerophosphocholine, alpha-lipoic acid, luteolin-7-glucoside, luteolin, quercetin, xylose, lutein, zeaxanthin, astaxanthin, meso-zeaxanthin, beta-carotene, zinc, copper, vitamin B1, vitamin B6, vitamin B12, vitamin A, vitamin C, vitamin D, vitamin E, and omega-3 fatty acids.

In another embodiment, the human subject is a healthy human subject, in particular an adult.

In yet another embodiment, the Aronia berry extract is an Aronia melanocarpa berry extract.

In a specific embodiment, the composition according to the invention is an oral composition.

In yet another specific embodiment, the composition is in the form of a pill, powder, capsule, tablet, gel, gummy, beverage, chewing gum, chewable tablet, lozenge or troche.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Retinal vasculature parameters after 1 week of Aronia extract supplementation in healthy subjects. (A) The arteriolar-to-venular ratio (A/V ratio) was significantly higher in the Brainberry/AME group compared to the control group. (B) The central retinal arteriolar equivalents (CRAE) was significantly higher in the Brainberry/AME group compared to the control group.

FIG. 2: BDNF levels after Aronia extract supplementation in healthy subjects. After one week of supplementation, an increase in BDNF levels was observed in the AME supplemented group.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The overall objective of the present invention is to provide a composition for non-therapeutic use to reduce, alleviate, improve symptoms related with several aspects of eye health including ocular blood flow, dry eyes and/or eye fatigue. These symptoms can be exacerbated by oxidative stress, chronic inflammation and/or hormonal disturbance. Where the composition of the present invention is particularly suitable for use in subjects wherein the above defined symptoms are perceptible or at least are experienced by the subject, it may also be suitably used in healthy subjects who do not perceive or experience such symptoms and thus may aid in the prevention of such symptoms or may even improve the eye condition.

As already mentioned hereinbefore, it was surprisingly found that an Aronia berry extract or a composition comprising an Aronia berry extract has beneficial effects on eye parameters. In particular, as described in example 2 of the present invention, one week of supplementation resulted in a significantly improved retinal micro vasculature as exemplified by a higher central retinal arteriolar equivalent (CRAE) and arteriolar/venular (A/V) ratio compared to a control group.

In a first aspect, the present invention provides the non-therapeutic use of an Aronia berry extract or a composition comprising said Aronia berry extract for improving ocular blood flow, improving symptoms of dry eyes, and/or improving symptoms of subjective eye fatigue in a human subject.

As used herein, the term “extract” refers to all active substances separated from a natural ‘raw material’ product by an appropriate solvent such as but not limited to ethanol, oil or water wherein all or nearly all of the solvent is evaporated after the extraction process. Extracts of Aronia berries may comprise fibers, vitamins, minerals, and other natural substances with antioxidant, lipid-lowering, and antiproliferative properties. The extract of the present invention contains all extracts and purified substances obtained in each step of extraction or purification, a diluted solution thereof, a concentrate or a dried product. Extracts may be in the form of tinctures, absolutes, syrups or in powder form.

As used herein and unless otherwise specified, the term “composition” refers to the identity of all different, individual substances, compounds or elements (e.g. agents, modulators, regulators, etc.) that constitute the composition, in combination with the Aronia berry extract. It can be a solution, mixture, an emulsion, a suspension, liquid, aqueous or non-aqueous composition prepared according to a specific procedure. Some components impart specific properties to the composition when it is put into use.

In particular, as defined herein, the term ‘composition’ is in particular used in the context of an Aronia berry extract that is further processed in a form that is particularly suitable for administration to a subject. Wherein the Aronia berry extract is a pure extract form that can be administered or used as a dried powder, tincture, absolute or syrup, the composition according to the invention can be processed in a more user-friendly dosage such as a capsule or pill for use. Therefore, when reference is made to a “composition”, it comprises an Aronia extract according to the invention and one or more compound to obtain such a composition. Accordingly, the terms “extract” and “composition” can be used interchangeably. For example, when reference is made to the non-therapeutic use of the Aronia berry extract, it is also meant to include the composition according to the invention, and vice versa, unless otherwise stated.

In yet another embodiment, the Aronia berry extract is an Aronia melanocarpa berry extract.

The extract according to the invention can be obtained by extracting, isolating and fractionating whole Aronia berries with or without the seeds, using extraction, separation and fractionation methods known in the art, and can be extracted according to various extraction solvents and extraction methods.

Exemplary methods for preparing Aronia berry extracts derived from Aronia melanocarpa may be found in KR101978336B1. Following such extraction methods, extracts in the form of a powder may be obtained. Alternatively, an extract in accordance with the present invention may also be in the form of a juice concentrate of Aronia berries with or without the seeds, wherein said juice concentrate may be a standardized concentrate.

As an illustrative example, an Aronia berry extract is prepared using a method comprising the following steps:
a) immersing Aronia berries with an extraction solution being water or an extraction solution essentially made of an alcoholic solution comprising at least 25% alcohol by weight, for example ethanol, for a period of time of at least 4 hours;
b) recovering the resultant extraction solution from end of step a);
c) immersing Aronia berries from step a) with a new extraction solution, which composition is similar to that defined hereabove, for a period of time of at least 4 hours;
d) recovering the extraction solution from end of step c);
e) collecting the resultant extraction solutions respectively from step b) and step c);
f) vacuum distillating the resultant solution from step e), for example at 70° C., so as to yield a concentrate comprising from 30% to 40% by weight of solids; and
g) drying the resultant concentrate from end of step f, for example by spraying at 70° C., so as to yield an extract as particles comprising from 10% to 40% by weight of anthocyanin content.

The solution for extracting the Aronia berry extract may include a crude extract, a polar solvent extract, or a non-polar solvent soluble extract, preferably a polar solvent extract. In addition, the polar solvent-soluble extract may be water or an organic solvent such as but not limited to methanol, n-propyl alcohol, isopropyl alcohol, and t-butyl alcohol. Or a mixture thereof.

The non-therapeutic use of the extract or composition according to the invention is specifically directed to human subjects, in particular adult human subjects. Within the context of the present invention, the term “subject” or “individual” or “participant” refers to a human, in particular an adult human. Preferentially, the subject is a human subject wherein the above defined symptoms are perceptible or at least experienced by the subject. However, the extract or composition described herein can also be applied in by subjects who do not perceive or experience any of the symptoms and thus can be applied as a non-therapeutic use in asymptomatic subject to prevent occurrence of such symptoms.

In another embodiment, the human subject is a healthy human subject, in particular an adult, more in particular an adult between the age of about 18 to about 100 years old, more in particular between about 18 and about 90 years old, even more in particular between about 18 and about 75 years old.

In the context of the present invention, the term ‘non-therapeutic use’ is to be understood as a non-therapeutic method to alleviate, improve or reduce symptoms that are related with a particular disease, illness or ailment, without the actual treatment or prevention of the disease as such. Substances or compounds can be used non-therapeutically in the context of cosmetic treatment. In the context of the present invention, the non-therapeutic use can also mean the use of substances or composition for purposes which cannot be properly understood as therapeutic, for example in the following circumstances:
a) where there is a disease or some form of ill health which has been correctly diagnosed and for which no pharmacological treatment is available;
b) where there is no specific and authorized prescription for drugs;
c) where the substance is used at dosages, in ways and for time periods which are in line with what has been established by scientific research as ‘effective and tolerable’ in to alleviate, reduce, or improve symptoms that are associated with an individual's particular illness.

As an example, an Aronia berry extract or composition comprising such an extract can be administered to a subject in an effective dosage, and optionally repeating said dosage, until a beneficial effect has occurred, without specifically treating a disease or ailment. Accordingly, the beneficial effect can also be a cosmetically beneficial effect.

As used herein, an “effective dosage” is an amount that prevents a deficiency, ameliorates a disease or medical condition in an individual or, more generally, reduces symptoms, manages progression of the disease or provides a nutritional, physiological, or medical benefit to the individual. The relative terms “improved,” “increased,” “enhanced”, “alleviated”, “reduce”, “ameliorate” and the like refer to the effects of a composition comprising an Aronia berry extract relative to a composition lacking an Aronia berry extract but otherwise identical (i.e. placebo or control). For example, the extract or composition according to the invention can reduce or improve disclosed symptoms by at least 0.1%, such as at least 0.5%, at least 1.0%, at least 1.5%, preferably at least 2.0%, more preferably at least 2.5%, and particularly preferably at least 3.0% when compared to untreated subjects. In another example, the extract or composition according to the invention can reduce or improve disclosed symptoms by at least 5%, such as at least 10%, at least 15%, at least 20%, preferably at least 25%, more preferably at least 30%, and particularly preferably at least 40%, when compared to untreated subjects.

A particular object of the present invention relates to the non-therapeutic use of an Aronia berry extract or a composition comprising said Aronia berry extract for improving ocular blood flow.

In a further embodiment, the improved ocular blood flow is the result of improved retinal vasculature, in particular improved retinal microvasculature.

As used herein, the term “retinal vasculature” is to be understood as the totality of retinal blood vessels in the inner retina whereas the term “retinal microvasculature” is to be understood as the thin capillaries that branch from the central retinal artery as well as the thin capillaries that converge to the central retinal vein. The retinal microvasculature can be used to assess microvascular function and thus the health of the eye and is a proxy for other small vessels within the body such as blood vessels in the brain. Thus, structural changes in the retinal vasculature are related to the development of eye diseases. Retinal vascular calibre can be analyzed through fundus images and can be measured in terms of central retinal artery equivalent (CRAE), central retinal vein equivalent (CRVE) and arterio-venous ratio (AVR). CRAE is a summary index reflecting the average width of retinal arterioles within a pre-defined diameter of the retina, and CRVE is a summary index reflecting the average width of retinal venules within a pre-defined diameter of the retina. CRAE and CRVE reflect distinct systemic vascular physiology and disease pathways that tend to target the arterial and venous systems specifically. It has been demonstrated that a narrower CRAE is associated with hypertension and age, indicating that a wider CRAE is related to a healthier phenotype. Low arteriole diameter and high venule diameter, both leading to a low A/V ratio, are associated with various cardiovascular complications. Furthermore, screentime has been associated with narrower retinal arteriolar caliber. A/V ratio is a dimensionless ratio of arteriole to venous calibre. However, AV ratio is non-specific, and changes in A/V ratio may reflect changes in arterioles or venules, or both. AN ratio is frequently used to represent generalized arteriolar narrowing and previous studies have shown that the A/V ratio is linked to ocular vascular diseases and thus eye health. As used herein, the A/V ratio is calculated using, the central retinal venous equivalent (CRVE) and the central retinal artery equivalent (CRAE) as CRAE/CRVE.

The inventors have unexpectedly found that the overall AN ratio and CRAE were significantly increased in an intervention group that received an Aronia berry extract supplementation compared to the control group (example 2). Since screentime is associated with narrower retinal arteriolar caliber (Gopinath B et. al. 2011), the extract or composition according to the invention provides a solution to this problem.

In a particular embodiment, the use of an extract or composition according to the invention can improve an ocular blood flow related symptom such as retinal vasculature, in particular retinal microvasculature by at least 0.1%, such as at least 0.5%, at least 1.0%, at least 1.5%, at least 2%, at least 3%, at least 4%, at least 5%, at least 6%, at least 7%, at least 8%, at least 9%, at least 10%, preferably at least 3%, more preferably at least 4%, and particularly preferably at least 5% when compared to untreated subjects.

For example, the use of an extract or composition according to the invention can improve retinal microvasculature in subjects by 5% compared to subjects who did not receive a composition according to the invention.

A further object of the present invention relates to the non-therapeutic use of an Aronia berry extract or a composition comprising said Aronia berry extract for improving symptoms of dry eyes.

In another embodiment, the dry eye symptoms are reduced tear production, increased tear osmolarity and/or tear film instability.

As used herein, the term “dry eye” refers to an eye condition that occurs when the eye is not adequately lubricated by tears. Tears can be inadequate and unstable for many reasons. For example, dry eyes may occur if tear production is inadequate or if tears are of poor-quality. This tear instability leads to inflammation and damage of the eye's surface. Other associated symptoms with dry eyes include irritation, burning, itching, stinging, pain, pulling sensation, redness, discharge, pressured eyed, blurred vision and easily fatigued eyes.

In a further embodiment, the dry eye symptoms are one or more symptoms selected from but not limited to the list comprising: reduced tear production, increased tear osmolarity, tear film instability, increased irritation, increased redness, increased discharge, increased blurred vision and/or easily fatigued eyes.

As used herein, the term “tear production” is to be understood as the formation of tear films by the tear gland or lacrimal gland. Tear production can be tested with for example a Schirmer's test to determine whether the eye produces enough tears to keep it moist. The test works by the principle of capillary action, which allows the water in tears to travel along the length of a paper test strip that is placed in the lower eye lid pouch. The rate of travel along the test strip is proportional to the rate of tear production. The Schirmer test score is determined by the length of the moistened area of the strips (using the scale packaged with the strips). For example, a person normally moistens 15 mm in 5 minutes of each paper strip while a person experiencing dry eyes might only wet 10 mm in 5 minutes.

As used herein, the term “tear osmolarity” is to be understood the concentration of salt in tears wherein the amount of proteins, lipids and mucins in tears remains stable. Thus, changes in osmolarity are due to fluctuations in water content, not tear composition specifically. In particular, hyperosmolarity, whether due to an increased evaporation rate or a reduction of tear secretion, is indicative of reduced aqueous levels. Tear osmolarity can be tested with for example an osmometer.

In a particular embodiment, the use of an extract or composition according to the invention can improve at least one dry eye related symptom selected from the above-mentioned list by at least 0.1%, such as at least 0.5%, at least 1.0%, at least 1.5%, at least 2%, at least 3%, at least 4%, at least 5%, at least 6%, at least 7%, at least 8%, at least 9%, at least 10%, preferably at least 3%, more preferably at least 4%, and particularly preferably at least 5% when compared to untreated subjects.

For example, the use of an extract or composition according to the invention can enhance tear production in subjects by at least 5%, 6%, 7%, 8%, 9%, 10% compared to subjects who did not receive a composition according to the invention.

In another embodiment, the use of an extract or composition according to the invention can reduce tear osmolarity in subjects by at least 5%, 6%, 7%, 8%, 9%, 10% compared to subjects who did not receive a composition according to the invention.

In another embodiment, the use of an extract or composition according to the invention can increase tear film stability in subjects by at least 5%, 6%, 7%, 8%, 9%, 10% compared to subjects who did not receive a composition according to the invention.

A further object of the present invention relates to the non-therapeutic use of an Aronia berry extract or a composition comprising said Aronia berry extract for improving symptoms of subjective eye fatigue.

In another embodiment, the symptoms of subjective eye fatigue symptoms are blurred vision at near distances, blurred vision at intermediate distances, blurred vision at far distances, difficulty or slowness in refocusing eyes from one distance to another, irritated eyes, burning eyes, dry eyes, eye strain, headache, tired eyes, sensitivity to bright lights, eye soreness, blurred vision, dry eye, foreign body sensation, and tearing.

As use herein, the term “eye fatigue”, also known as asthenopia or eye strain, is to be understood as an eye condition that manifests through non-specific symptoms such as fatigue, pain in or around the eyes, blurred vision at near distances, blurred vision at intermediate distances, blurred vision at far distances, headache, occasional double vision, difficulty or slowness in refocusing, dry eyes, eye discomfort, headaches, irritated or burning eyes, sensitivity to bright lights, tired eyes and/or sore eyes. Symptoms often occur after long-term use of computers, digital devices, reading or other activities that involve extended visual tasks which are broadly classified into external and internal symptom factors. Self-reported eye fatigue symptoms can be assessed via a questionnaire.

In a particular embodiment, the use of an extract or composition according to the invention can improve at least one eye fatigue related symptom selected from the above-mentioned list by at least 0.1%, such as at least 0.5%, at least 1.0%, at least 1.5%, at least 2%, at least 3%, at least 4%, at least 5%, at least 6%, at least 7%, at least 8%, at least 9%, at least 10%, preferably at least 3%, more preferably at least 4%, and particularly preferably at least 5% when compared to untreated subjects.

For example, the use of an extract or composition according to the invention can reduce eye soreness in subjects by 5%, 6%, 7%, 8%, 9%, 10% compared to subjects who did not receive a composition according to the invention.

Thus, the present application relates to the non-therapeutic use of an Aronia berry extract or a composition comprising said Aronia berry extract. Aronia berries contain a large amount of flavonoid (polyphenolic compounds) such as anthocyanins. In the following paragraphs, concentrations of anthocyanins and other compounds are disclosed and relate to concentrations in the Aronia berry extract. One skilled in the art will understand that final concentrations in the composition comprising the Aronia extract will deviate proportionally to the amount of extract used in the composition.

As used herein, the term “anthocyanin” also termed “anthocyans” refer a parent class of molecules called flavonoids synthesized via the phenylpropanoid pathway. Anthocyanins are water-soluble vacuolar pigments that in all tissues of higher plants, including leaves, stems, roots, flowers, and fruits and depending on their pH, may appear red, purple, blue, or black. Thus, anthocyanins, are biosynthesized in plants by regulatory genes that encode key enzymes involved in the flavonoid biosynthetic pathway and regulatory genes that regulate the expression of these structural genes. Flavonoid compounds such as anthocyanins protect plants against insects and external stress, and act as antioxidants to prevent damage by active oxygen. In particular, when these anthocyanins are ingested into the animal body, they protect the somatic cells through the antioxidant action, enhance the immune system, and exhibit excellent anticancer activity. Furthermore, it is known that anthocyanin has an anticancer activity in various cancer cells and has an inhibitory effect of NF-κB which is known to be involved in cancer growth, metastasis and tolerance.

In a specific embodiment, the Aronia berry extract comprises at least 10% (w/w) anthocyanins, preferably at least 15% (w/w) anthocyanins, more preferably at least 20% (w/w) anthocyanins, even more preferably at least 25% (w/w) anthocyanins.

In a specific embodiment, the Aronia berry extract comprises about and between 1% to about 50% (w/w), preferably about and between 5% to about 40% (w/w), more preferably about and between 10% to about 35% (w/w), most preferably about and between 15% and about 30% (w/w) of anthocyanin.

In another specific embodiment, the Aronia berry extract comprises about and between about 5%, 10%, 15%, 20%, 25%, 30%, 35%, 40%, 45%, 50%, 55%, 60%, 65%, 70%, 75%, 80% (w/w) of anthocyanins.

In another specific embodiment, the anthocyanin is a cyanidin-3-O-glycoside selected from the list comprising: cyanidin-3-galactoside, cyanidin-3-glucoside, cyanidin-3-arabinoside, cyanidin-3-xyloside or any combination thereof.

In a specific embodiment, the extract or composition according to the invention comprises cyanidin-3-galactoside in combination with cyanidin-3-glucoside; alternatively cyanidin-3-galactoside in combination with cyanidin-3-arabinoside; alternatively cyanidin-3-galactoside in combination with cyanidin-3-xyloside; alternatively cyanidin-3-glucoside in combination with cyanidin-3-arabinoside; alternatively cyanidin-3-glucoside in combination with cyanidin-3-xyloside; alternatively cyanidin-3-arabinoside in combination with cyanidin-3-xyloside; alternatively cyanidin-3-galactoside in combination with cyanidin-3-glucoside, cyanidin-3-xyloside; alternatively cyanidin-3-galactoside in combination with cyanidin-3-arabinoside, cyanidin-3-xyloside; alternatively cyanidin-3-glucoside in combination with cyanidin-3-arabinoside, cyanidin-3-xyloside; alternatively cyanidin-3-galactoside in combination with cyanidin-3-glucoside, cyanidin-3-arabinoside, cyanidin-3-xyloside.

In yet another embodiment, in the case that cyanidin-3-galactoside is present in the Aronia berry extract or a composition comprising said Aronia berry extract, then cyanidin-3-galactoside comprises at least 30% (w/w), preferably at least 40% (w/w), more preferably at least 50% (w/w), most preferably at least 60% (w/w) of the total amount of cyanidin-3-O-glycoside.

In a specific embodiment, in the case that cyanidin-3-galactoside is present in the Aronia berry extract or a composition comprising said Aronia berry extract, then cyanidin-3-galactoside comprises about and between 25% to about 100% (w/w), preferably about and between 35% to about 90% (w/w), more preferably about and between 40% to about 80% (w/w), most preferably about and between 50% and about 70% (w/w) of the total amount of cyanidin-3-O-glycoside.

In another specific embodiment, in the case that cyanidin-3-galactoside is present in the Aronia berry extract or a composition comprising said Aronia berry extract, then cyanidin-3-galactoside comprises about 20%, 25%, 30%, 35%, 40%, 45%, 50%, 55%, 60%, 65%, 70%, 75%, 80% (w/w) of the total amount of cyanidin-3-O-glycoside.

In a particular embodiment, the Aronia berry extract comprises at least about 5% (w/w) cyanidin-3-galactoside, preferably at least about 10% (w/) cyanidin-3-galactoside, more preferably at least about 15% (w/w) cyanidin-3-galactoside (w/w) of the total amount of the extract.

In a specific embodiment, the Aronia berry extract comprises about and between 5% to about 80% (w/w), preferably about and between 10% to about 70% (w/w), more preferably about and between 15% to about 60% (w/w), most preferably about and between 20% and about 50% (w/w) cyanidin-3-galactoside of the total amount of the extract.

In another specific embodiment, the Aronia berry extract comprises about 5%, 10%, 15%, 20%, 25%, 30%, 35%, 40%, 45%, 50%, 55%, 60%, 65%, 70%, 75%, 80% (w/w) cyanidin-3-galactoside of the total amount of the extract.

In a particular embodiment, the Aronia berry extract comprises at least about 3% (w/w), at least about 5% (w/w) cyanidin-3-galactoside, preferably at least about 10% (w/) cyanidin-3-glucoside, more preferably at least about 15% (w/w) cyanidin-3-galactoside (w/w) of the total amount of the extract.

In a specific embodiment, the Aronia berry extract comprises about and between 5% to about 80% (w/w), preferably about and between 10% to about 70% (w/w), more preferably about and between 15% to about 60% (w/w), most preferably about and between 20% and about 50% (w/w) cyanidin-3-glucoside of the total amount of the extract.

In another specific embodiment, the Aronia berry extract comprises about 5%, 10%, 15%, 20%, 25%, 30%, 35%, 40%, 45%, 50%, 55%, 60%, 65%, 70%, 75%, 80% (w/w) cyanidin-3-glucoside of the total amount of the extract.

In a further embodiment, the extract of the invention comprises:
at least 10% anthocyanins;
of which at least 25% cyanidin-3-O-glycosides; and
of which at least 50% cyanidin-3-galactoside.

In a further embodiment, the extract of the invention comprises:
at least 20% anthocyanins;
of which at least 25% cyanidin-3-O-glycosides; and
of which at least 50% cyanidin-3-galactoside.

It has been observed that such a combination is particularly suitable to improve ocular blood flow, reduce dry eye symptoms and improve and eye fatigue.

In another particular embodiment, the composition of the present invention comprises about and between 1 mg to 1000 mg Aronia berry extract, such as about and between 15 mg to 750 mg, about and between 30 mg to 750 mg of Aronia berry extract, about and between 65-750 mg Aronia berry extract, about and between 90-750 mg Aronia berry extract, about and between 65-500 mg Aronia berry extract, about and between 65-250 mg Aronia berry extract, or about and between 65-100 mg Aronia berry extract.

In another embodiment, the composition comprises at least 15 mg of Aronia berry extract, such as at least 30 mg, at least 50 mg, at least 100 mg, at least 125 mg or at least 200 mg of Aronia berry extract.

In an even further embodiment, the composition according to the invention comprises about 5 mg, 10 mg, 15 mg, 25 mg, 50 mg, 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg of Aronia berry extract.

In yet another particular embodiment, the composition comprises about and between 1 mg to 500 mg, preferably about and between 10 mg to 300 mg, more preferably about and between 15 mg to 200 mg of anthocyanin.

In another embodiment, the composition comprises at least 5 mg of anthocyanin, such as at least 10 mg, at least 15 mg, at least 20 mg, at least 30 mg or at least 40 mg of anthocyanin.

In an even further embodiment, the composition according to the invention comprises about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 140 mg, about 160 mg, about 180 mg, about 200 mg of anthocyanin.

In a specific embodiment, the composition of the invention comprises an Aronia berry extract comprising about 16% or about 25% of anthocyanin.

A non-limiting example of a composition according to the invention comprises about 65 mg of Aronia berry extract of which about 16 mg is anthocyanin.

Another non-limiting example of a composition according to the invention comprises about 200 mg of Aronia berry extract of which about 50 mg is anthocyanin.

Another non-limiting example of a composition according to the invention comprises about 250 mg of Aronia berry extract of which about 60 mg is anthocyanin.

Another non-limiting example of a composition according to the invention comprises about 300 mg of Aronia berry extract of which about 75 mg is anthocyanin.

Another non-limiting example of a composition according to the invention comprises about 750 mg of Aronia berry extract of which about 180 mg is anthocyanin.

In some embodiments, the compositions may further comprise optionally one or more of a protein source, a free amino acid, a carbohydrate source, a fat source, a vitamin, or a mineral.

In an embodiment, the composition comprising the Aronia berry extract further comprises one or more compounds selected from a group consisting of: caffeine, taurine, theanine, inositol, guarana, magnesium, melatonin, ribose, carnitine, co-enzym Q10, alpha-glycerophosphocholine, alpha-lipoic acid, luteolin-7-glucoside, luteolin, quercetin, xylose, lutein, zeaxanthin, astaxanthin, meso-zeaxanthin, beta-carotene, zinc, copper, vitamin B1, vitamin B6, vitamin B12, vitamin A, vitamin C, vitamin D, vitamin E, and omega-3 fatty acids.

In an embodiment, the extract or composition according to the invention can be administered in multiple doses per day and more specifically at least once, twice, three times, four times a day, preferably in at least one or two doses per day.

In a preferred embodiment, the composition is administered on a regimen of one intake per day.

In a particular embodiment, the composition is administered on a regimen of one intake per day comprising about 65 mg of Aronia berry extract, with about 16 mg of anthocyanin.

In a particular embodiment, the composition is administered on a regimen of three intakes per day wherein each intake comprises about and between 150 mg to 500 mg of Aronia berry extract, in particular about 250 mg of Aronia berry extract.

In another particular embodiment, the composition is administered on a regimen of three intakes per day wherein each intake comprises about and between 15 mg to 500 mg, preferably 60 mg of anthocyanins.

In another embodiment, the composition can also be administered on in 1 intake per day wherein said composition comprises about 750 mg of Aronia extract of which about 180 mg anthocyanins.

In an embodiment, the composition according to the invention is used for human and/or animal consumption.

In a specific embodiment, the composition according to the invention is an oral composition.

The composition is administered as a supplement to the diet of an individual multiple times a week, more specifically once, twice, three times, four times, five times, six times, seven times a week, preferably daily.

In an embodiment, the composition is administered to the individual consecutively for a number of days until a beneficial effect is achieved. For example, the composition can be administered to the individual daily for at least 10, 20, 30, 40, 50, 60, 70, 80, 90, 100, 110, 120 or more consecutive days. As another example, the composition can be administered to the individual for a longer period, such as a period of 1, 2, 3, 4, 5, 6, 7, 8, 9 or 10 years.

In a particular embodiment, the composition is administered to the individual for at least 1 week, such as at least 2 weeks, at least 3 weeks, at least 4 weeks, preferably at least 1 weeks.

In another embodiment, the composition is administered at least 3 months, for example a period of 3 months to 1 year, and preferably for at least 6 months.

The above examples of administration do not require continuous daily administration with no interruptions. Instead, there may be some short breaks in the administration, such as a break of two to four days during the period of administration. The ideal duration of the administration of the composition can be determined by those of skill in the art.

The compositions for non-therapeutic use of the present invention, can be prepared in any known or otherwise effective dosage or product form suitable for use in providing to systemic delivery of the Aronia extract compounds to the site of interest, which would include both non-pharmaceutical dosage forms as well as nutritional product forms. For example, the extracts can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form.

Non-limiting examples of suitable excipients, diluents, and carriers can be: fillers and extenders such as starch, sugars, mannitol, and silicic derivatives; binding agents such as carboxymethyl cellulose and other cellulose derivatives, alginates, gelatin, and polyvinyl pyrolidone; disintegrating agents such as calcium carbonate and sodium bicarbonate; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

The compositions are preferably administered as oral dosage forms. For example, the composition may be selected from the group consisting of food compositions, dietary supplements, nutritional compositions, nutraceuticals, powdered nutritional products to be reconstituted in a liquid before consumption, food additives, medicaments, beverages and drinks. Preferred dosage or product forms in this respect include oral tablets, capsules, oral liquids as well as powders to be reconstituted in a liquid or to be used in for example but not limited to bakery matrices such as bread, cookies and biscuits; and dairy products such as butter, cheese, milk and yoghurt.

In yet another specific embodiment, the composition is in the form of a pill, powder, capsule, tablet, gel, gummy, beverage, chewing gum, chewable tablet, lozenge or troche.

In a preferred embodiment, the composition is in the form of a pill or capsule.

In another embodiment, the composition is in the form of a powder to be reconstituted in or sprinkled over food matrices. Suitable food matrices to reconstitute are but not limited to liquids such as water, milk, juices; gels such as grape jelly, emulsions such as mayonnaise, fibrous materials such as fruits and vegetables, crystals such as sugars, porous structures such as marshmallows and much more.

In another particular embodiment, the present invention provides a method for improving ocular blood flow, improving symptoms of dry eyes, and/or improving symptoms of subjective eye fatigue in a human subject, the method comprising administering an Aronia berry extract or a composition comprising an Aronia berry extract to the subject.

### EXAMPLES

### Example 1—Detailed Protocol on Eye Health Parameters

### Methods

### Study Population

Healthy 18-35-year-old adults with a body mass index (BMI) between 18-30 kg/m² were recruited through local advertisements and social media. Exclusion criteria included severe medical conditions, use of medication or supplements that might influence endpoints of the study, high blood pressure, smoking, and abuse of products (>3 alcoholic units/day and drug use).

### Study Design

The present study is a randomized, double-blind, placebo-controlled, cross-over trial consisting of two study groups and a pre- and post-test day in both study arms. Participants received capsules containing either 65 mg of the Aronia melanocarpa extract (AME) or the placebo in a randomized order, each for 6 weeks, separated by a wash-out period of 6 weeks. At baseline and after 6 weeks of intervention, participants visited the research facilities for study measurements. From two days prior to blood sampling, participants were asked not to perform any strenuous physical exercise or to consume alcoholic beverages. Furthermore, subjects were asked not to change their habitual diet prior to the test days and to avoid anthocyanin-rich foods.

### Intervention

The nutritional supplement used in this project is an AME containing 25% anthocyanins (Brainberry, BioActor B.V., Maastricht, The Netherlands). These dietary anthocyanins consist of several cyanidin-3-glycosides, such as cyanidin-3-galactoside, cyanidin-3-arabinoside, cyanidin-3-xyloside, and cyanidin-3-glucoside. Intervention capsules contained either 65 mg AME or 65 mg cellulose (placebo). The provided AME capsules consist of 16 mg anthocyanins. Participants were instructed to ingest one capsule daily before breakfast, with 200 mL water.

### Measurements

Participants underwent measurements in the morning in a fasted state in a room with controlled temperature at 22° C. On the pre-measurement days, anthropometric measurements were conducted, and a blood sample was collected for cognitive performance biomarker analysis of BDNF. Moreover, microvascular function through fundus photography, and perceivable benefits was assessed, including general well-being (quality of life), sleep characteristics: the duration, quality, latency and efficiency, and mood: the degree of pleasantness and arousal. Moreover, eye health was assessed by a tear production test (Schirmer's test) and a subjective eye fatigue questionnaire.

On the post-measurement days, next to the abovementioned measurements, cognitive performance was be assessed by a digital neuropsychological test battery (CANTAB). Participants also completed a food diary twice.

### Schirmer's Test

Tear sampling was performed by adsorption of tear fluid in a paper (Schirmer) strip. A Schirmer strip is a special paper strip which is placed in de lower eyelid of the subject for 5 minutes. It is a standard method to evaluate the level of tear production of the eyes in order to diagnose dry eyes disease. After 5 minutes, the Schirmer strip is gently removed by the ophthalmological researcher using tweezers and stored at −80° C. During this measurement, the subject was in a comfortable sitting position. The burden on the subject is minimal. The procedure took 10 minutes in total (5 minutes per eye). The maximal tear sample volume was approximately 35 μL per eye. The samples were stored at −80 C for future analysis of biomarkers related to dry eye, only if changes in the Schirmer's test were observed.

### Osmolarity Measurement

Osmolarity measurement were performed within the first 30 min after video display terminal. Tear film osmolarity (TFO) was measured using the I-PEN® osmometry system, which includes single-use sensors that measure TFO by electrical impedance. The test was performed on both eyes according to the manufacturer's specifications, touching the tear meniscus with the sensor near the lateral canthus. The mean of three consecutive measurements were calculated to increase the accuracy and repeatability.

### Subjective Eye Fatigue Questionnaire

Before and after VDT loading, subjective eye fatigue was assessed using an eye symptom questionnaire developed by Hayes et al. 2007.

### Microvascular Function

Retinal images were obtained to visualize the response of the microvasculature. For this, study participants were seated with the head resting on a chinrest, looking directly into the camera (Topcon TRC-NW-300, Topcon Corporation, Tokyo, Japan) while a picture from the retina was taken. The total procedure took about 2 minutes. Images were digitized and analyzed to calculate the Arteriole-to-Venule-ratio with the appropriate software (Generalized Dual-Bootstrap Iterative Closest Point (GDB-ICP)) that is able to automatically initialize and individually match vascular landmarks. In addition, the software was used to measure the diameter of the four largest retinal arterioles and venules).

### Example 2—Effects in Healthy Subjects

### Methods

Healthy 18-35-year-old adults with a body mass index (BMI) between 18.5-30 kg/m² were recruited through local advertisements and social media. Exclusion criteria were pre-existing neurological illnesses, use of medication that might influence endpoints of the study (such as antidepressants or blood pressure medication), high blood pressure, vitamin, mineral, or antioxidant supplementation, pregnancy, smoking, and abuse of products (>20 alcoholic units/week and drug use).

### Study Design

A randomized, double-blind, placebo-controlled, cross-over study was performed. After successful screening, each participant was randomly allocated to receive one week of either Aronia melanocarpa extract (AME) or placebo, separated by a wash-out period of at least two weeks. At baseline and after one week of intervention, participants visited the research facility and fundus photography and blood collection were performed in temperature-controlled rooms.

During the study, participants were asked to refrain from foods containing berry polyphenols. Furthermore, participants were instructed to refrain from strenuous physical activity and alcohol on the two days preceding each test day, and to arrive fasted to the research facility.

### Intervention

The study product was an Aronia melanocarpa extract (AME) containing 24% anthocyanins (cyanidin-3-galactoside, cyanidin-3-arabinoside, cyanidin-3-xyloside, cyanidin-3-glucoside) (Brainberry®, BioActor BV, Maastricht, The Netherlands). 750 mg AME was provided in three capsules containing 180 mg anthocyanins/day in total. Cellulose-containing capsules were used as placebo. Participants were instructed to consume three capsules daily before breakfast, with 200 mL water.

### Measurements

Participants underwent measurements in the morning in a fasted state in a room with controlled temperature at 22° C. On the pre-measurement days, anthropometric measurements were conducted, and a blood sample was collected for cognitive performance biomarker analysis of BDNF. Moreover, microvascular function was assessed through fundus photography.

### Results

### Baseline Characteristics

The mean age was 25 years old, with a BMI of 23.4 kg/m². All baseline characteristics are presented in Table 1.

TABLE 1
Baseline characteristics of study participants at baseline¹.

| | Study participants (n = 35) |
|---|---|
| Male gender (n; %) | 16; 46% |
| Age (years) | 25 ± 4 |
| BMI (kg/m²) | 23.4 ± 2.7 |
| Systolic blood pressure (mm Hg) | 106 ± 2 |
| Diastolic blood pressure (mm Hg) | 68 ± 1 |

¹Data is displayed as mean ± standard deviation or as a percentage.

### Fundus Photography

Changes in retinal vascular caliber can provide important information about the state of the retinal microcirculation. Microvascular calibers were determined using a retinal fundus camera. Retinal vascular images of the right optic disc were taken and analyzed using Interactive Vessel Analyzer (IVAN) software. Using the Parr-Hubbar formula, the mean central retinal arteriolar and venular equivalents (CRAE and CRVE, respectively) and the arteriolar-to-venular ratio (A/V ratio) were calculated. The A/V ratio reflects the diameter of the arteriole and the venule. Low arteriole diameter and high venule diameter, both leading to a low A/V ratio, are associated with various cardiovascular complications. Furthermore, screentime has been associated with narrower retinal arteriolar caliber.

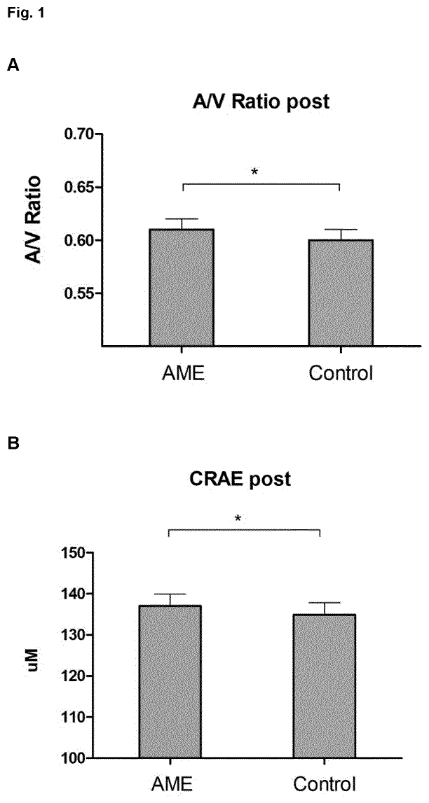

After one week of supplementation, the A/V ratio was significantly higher in the Brainberry/AME group compared to the control group (p=0.001) (FIG. 1A).

After one week of supplementation, the central retinal arteriolar equivalents (CRAE) was significantly higher in the Brainberry/AME group compared to the control group (p=0.011) (FIG. 1B).

### BDNF Levels

Brain-derived neurotrophic factor (BDNF) is considered a key molecule for cognition and eye health in particular dry eyes. In Bdnf knockdown mice, a decreased basal tear secretion and loss of stress tolerance by enriched environment for tear secretion has been shown suggesting that Bdnf expression is related to tear secretion (Sano et al. 2019). Low serum levels of BDNF are also involved in various eye disease pathologies such as macular degeneration, retinopathy, myopia and glaucoma. Furthermore, BDNF has been linked with neuroprotection in the retina. Maintenance or increase of BDNF levels is beneficial in the prevention of such pathologies or reduction of function.

BDNF levels were determined using a commercially available sandwich enzyme-linked immunosorbent assay (ELISA) kit, according to the manufacturer's protocol (Duoset, R&D systems, Bio-techne, Minneapolis, MN, USA).

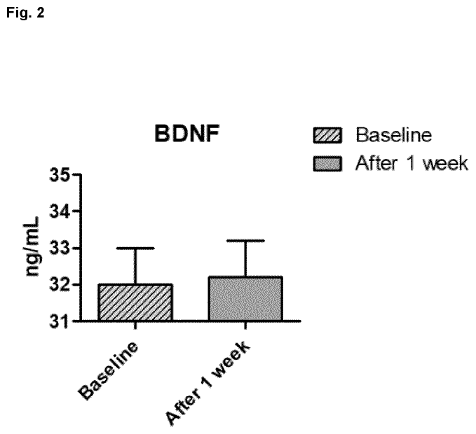

An increase in BDNF levels was observed after 1 week in the group that received AME supplementation (FIG. 2). BDNF plays an important role in maintenance of eye health, as negative effects of blue light and other eye conditions have been related to a decrease in BDNF levels. Therefore, an increase in BDNF can ameliorate these negative effect and improve eye health.

### REFERENCES

Ahles, S.; Stevens, Y. R.; Joris, P. J.; Vauzour, D.; Adam, J.; de Groot, E.; Plat, J. The Effect of Long-Term Aronia melanocarpa Extract Supplementation on Cognitive Performance, Mood, and Vascular Function: A Randomized Controlled Trial in Healthy, Middle-Aged Individuals. Nutrients 2020, 12, 2475.
Gopinath B, Baur L A, Wang J J, Hardy L L, Teber E, Kifley A, Wong T Y, Mitchell P. Influence of physical activity and screen time on the retinal microvasculature in young children. Arteriosclerosis, thrombosis, and vascular biology. 2011 May; 31(5):1233-9.
Kan, J.; Wang, M.; Liu, Y.; Liu, H.; Chen, L.; Zhang, X.; Huang, C.; Liu, B. Y.; Gu, Z.; Du, J. A novel botanical formula improves eye fatigue and dry eye: a randomized, double-blind, placebo-controlled study. Am J Clin Nutr 2020, 112, 334-342, doi:10.1093/ajcn/nqaa139.
Riva, A.; Togni, S.; Franceschi, F.; Kawada, S.; Inaba, Y.; Eggenhoffner, R.; Giacomelli, L. The effect of a natural, standardized bilberry extract (Mirtoselect®) in dry eye: a randomized, double blinded, placebo-controlled trial. Eur Rev Med Pharmacol Sci 2017, 21, 2518-2525.
Sano, K., Kawashima, M., Imada, T. et al. Enriched environment alleviates stress-induced dry-eye through the BDNF axis. Sci Rep 9, 3422 (2019).

1. Non-therapeutic use of an Aronia berry extract or composition comprising said Aronia berry extract for improving ocular blood flow, improving symptoms of dry eyes, and/or improving symptoms of subjective eye fatigue in a human subject.

2. Non-therapeutic use according to claim 1, wherein said improved ocular blood flow is the result of improved retinal vasculature, in particular improved retinal microvasculature.

3. Non-therapeutic use according to claim 1, wherein said dry eye symptoms are reduced tear production, increased tear osmolarity and/or tear film instability.

4. Non-therapeutic use according to claim 1, wherein said subjective eye fatigue is blurred vision at near distances, blurred vision at intermediate distances, blurred vision at far distances, difficulty or slowness in refocusing eyes from one distance to another, irritated eyes, burning eyes, dry eyes, eye strain, headache, tired eyes, and/or sensitivity to bright lights,

5. Non-therapeutic use according to any one of claims 1 to 4, wherein said Aronia berry extract comprises at least 10% (w/w) anthocyanins, preferably at least 15% (w/w) anthocyanins, more preferably at least 20% (w/w) anthocyanins, even more preferably at least 25% (w/w) anthocyanins.

6. Non-therapeutic use according to claim 5, wherein said anthocyanin is a cyanidin-3-O-glycoside selected from the list comprising: cyanidin-3-galactoside, cyanidin-3-glucoside, cyanidin-3-arabinoside, cyanidin-3-xyloside or any combination thereof.

7. Non-therapeutic use according to claim 5, wherein in the case that cyanidin-3-galactoside is present, cyanidin-3-galactoside comprises at least 30% (w/w), preferably at least 40% (w/w), more preferably at least 50% (w/w), most preferably at least 60% (w/w) of the total amount of cyanidin-3-O-glycoside.

8. Non-therapeutic use according to any one of claims 1 to 7, wherein the Aronia berry extract comprises at least about 5% (w/w) cyanidin-3-galactoside, preferably at least about 10% (w/) cyanidin-3-galactoside, more preferably at least about 15% (w/w) cyanidin-3-galactoside (w/w) of the total amount of the extract.

9. Non-therapeutic use according to any one of claims 1 to 8, wherein said composition comprises about and between 1 mg to 1000 mg of Aronia berry extract, such as about and between 15 mg to 750 mg, about and between 30 mg to 750 mg, about and between 65-750 mg of Aronia berry extract, or about and between 90-750 mg of Aronia berry extract.

10. Non-therapeutic use according to any one of claims 1 to 8, wherein said composition comprises about and between 1 mg to 500 mg, preferably about and between 10 mg to 300 mg, more preferably about and between 15 mg to 200 mg of anthocyanin.

11. Non-therapeutic use according to any one of claims 9 to 10, wherein said composition further comprises one or more compounds selected from a group consisting of: caffeine, taurine, theanine, inositol, guarana, magnesium, melatonin, ribose, carnitine, co-enzym Q10, alpha-glycerophosphocholine, alpha-lipoic acid, luteolin-7-glucoside, luteolin, quercetin, xylose lutein, zeaxanthin, astaxanthin, meso-zeaxanthin, beta-carotene, zinc, copper, vitamin B1, vitamin B6, vitamin B12, vitamin A, vitamin C, vitamin D, vitamin E, and omega-3 fatty acids.

12. Non-therapeutic use according to any one of claim 1 to 11, wherein said human subject is a healthy human subject, in particular an adult subject.

13. Non-therapeutic use according to any one of claim 1 to 12, wherein said Aronia berry extract is an Aronia melanocarpa berry extract.

14. Non-therapeutic use according to any one of claims 1 to 13, wherein said composition is an oral composition.

15. Non-therapeutic use according to any one of claims 1 to 14, wherein the composition is in the form of a pill, powder, capsule, tablet, gel, gummy, beverage, chewing gum, chewable tablet, lozenge or troche.
